(19) United States
(12) Patent Application Publication
(10) Pub. No.: US 2009/0291975 A1
(43) Pub. Date: Nov. 26, 2009

(54) DUAL OPIOID PAIN THERAPY

(76) Inventors: WARREN STERN, Plymouth, MA (US); Gary W. Pace, La Jolls, CA (US); Felix A. de la IgLesia, Ann Arbor, MI (US); Maree Smith, Bardon (AU); Patricia Richards, West New York, NJ (US)

Correspondence Address: KING & SPALDING, 1180 PEACHTREE STREET , NE, ATLANTA, GA, 30309-3521, US

(21) Appl. No.: 12/469,438

(22) Filed: May 20, 2009

Related U.S. Application Data
(60) Provisional application No. 61/128,246, filed on May 20, 2008.
(60) Provisional application No. 61/143,863, filed on Jan. 12, 2009.

(51) Int. Cl. A61K 31/485; A61P 29/00

(52) U.S. Cl. 514/282

(57) ABSTRACT

Provided are pharmaceutical compositions and methods for the alleviation of pain in a patient with optimal ratios of morphine and oxycodone that provide superior analgesic efficacy and lower incidence of adverse side effects compared to morphine and oxycodone alone. The pharmaceutical compositions comprise morphine and oxycodone, or pharmaceutically acceptable salts thereof, in ratios of about 3 to 2 to about 1 to 2, morphine to oxycodone by weight.

### CROSS REFERENCE TO RELATED APPLICATIONS

This application claims the benefit of U.S. Provisional Application No. 61/128,246 filed May 20, 2008, and U.S. Provisional Application No. 61/143,863, filed Jan. 12, 2009, both of which are incorporated herein by reference in their entirety.

### FIELD OF THE INVENTION

This invention is directed to pharmaceutical compositions comprising optimal combinations of morphine and oxycodone that provide synergistic efficacy and lower incidence of undesired side effects for patients undergoing pain therapy. Methods of use comprising administering an effective amount and ratio of the opioid compounds to treat patients suffering from pain are also provided.

### BACKGROUND OF THE INVENTION

Opioid compounds remain key agents for the treatment of a wide variety of acute and chronic pain. The World Health Organization has recommended morphine as the analgesic of choice for the treatment of severe cancer pain. Additionally, morphine and related opioids are widely used to alleviate moderate to severe pain after surgery or trauma, or associated with medical illness. Patients with apparently similar pain states can have large differences in opioid dosing requirements. Factors that contribute to this variability include psychosocial status, type of pain (nociceptive, inflammatory, neuropathic or mixed) and its severity, concurrent medications, gender and other genetic aspects, and whether patients are opioid-naïve or tolerant.

Unfortunately, the effects produced by morphine and similar opioid compounds are associated with many undesirable side effects, all mediated through activation of the mu and other opioid receptors, and make them amenable to abuse. The undesirable side effects associated with the use of opioids include nausea and vomiting, drowsiness, dizziness, constipation, respiratory depression and bladder dysfunction. Also physical and psychological dependence leading to addiction and other diverse pathophysiological states can occur.

Further a major associated risk is that repeated daily administrations of morphine or morphine-like opioids will eventually induce significant tolerance to the therapeutic effects of the drug, as well as initiating some degree of physical dependence. Opioid tolerance is a phenomenon whereby chronic exposure to a drug diminishes its antinociceptive or analgesic effect, or creates the need for a higher dose to maintain its effect. The degree of tolerance and physical dependence will vary with the particular opioid employed, the correlation with morphine opioid receptor-selective opioids, the frequency of administration, and the quantity of opioid administered.

In a wide variety of clinical indications requiring prolonged use of opioids, tolerance induction and addiction are closely linked, with the development of physical and psychological dependence always a major concern. Addiction with physical dependence can be difficult to treat due to the effects of withdrawal associated with dependence. Another undesirable effect of opioid tolerance is that the higher opioid requirements of highly tolerant patients treated for pain increase the likelihood of unpleasant non-analgesic side effects due to greater circulating concentrations of opioids and potentially toxic opioid metabolites (Smith, M. T., Clin. Exp. Pharmacol. Physiol. 2000, 27, 524-528; Ross et al., Pain, 1997, 73, 151-157).

The opioid receptor is thought to have four receptor subtypes named mu (morphine receptor), sigma (the phencyclidine receptor), kappa (the ketocyclazocine receptor) and delta (the endorphin/enkephalin receptor). The biochemical and cellular effects of morphine, including analgesia, are transduced through the mu opioid receptor (MOR), found in high concentrations within the central nervous system (CNS). The World Health Organization's guidelines for the management of chronic cancer pain recommend that clinicians reserve strong opioids, such as oxycodone and morphine, for the relief of moderate to severe cancer pain (World Health Organization, 1986). The guidelines also recommend that two strong opioids should not be co-administered, presumably because it is generally thought that all opioids exert their analgesic effects through the same receptor mechanisms in the central nervous system. However, studies by Maree Smith and co-workers have shown that the antinociceptive effects of structurally related oxycodone and morphine are differentially antagonized by nor-BNI (a κ-selective opioid antagonist) and naloxonazine (a selective μ-opioid receptor antagonist), indicating that they produce antinociception through different opioid receptor mechanisms (see Ross, F. B; Smith, M. T., Pain 1997, 73, 151-157). Furthermore, it has been found that co-administration to rats of sub-antinociceptive (also termed sub-analgesic) doses of oxycodone with morphine results in synergistic levels of antinociception (Ross et al., Pain 2000, 84, 421-428). It was found that animals that received the sub-antinociceptive doses of oxycodone and morphine were similar to vehicle injected control animals with respect to CNS side effects. Administration of equipotent-doses of either opioid alone resulted in sedation of the rats.

U.S. Pat. No. 6,310,072 to Smith et al. (incorporated herein in its entirety by reference) discloses analgesic compositions comprising a sub-analgesic dosage of a mu opioid agonist selected from the group consisting of morphine, fentanyl, sufentanil, alfentanil and hydromorphone, or a pharmaceutically acceptable salt thereof, and a sub-analgesic dosage of oxycodone which is a kappa-opioid agonist or a pharmaceutically acceptable salt thereof. Smith et al. disclose dosing regimens in terms of mg of therapeutic composition per kg of patient body weight over varying periods of time, but does not disclose a ratio of morphine and oxycodone that provide synergistic analgesia to human patients with reduction of opioid-related side effects. In particular, Smith et al. describe the administration of different combinations of morphine and oxycodone to male Sprague-Dawley and male Dark Agouti rats via intracerebroventricular (i.c.v.), intraperitoneal (i.p.) or subcutaneous (s.c.) administration, and the administration of a subanalgesic dose of morphine plus oxycodone (2.0 mg each) via intravenous administration. Smith et al. describes the use of a combination of sub-analgesic doses of morphine and oxycodone. Smith et al. defined the term ‘sub-analgesic dosage’ as a “dosage of a mu-opioid agonist solus or a kappa₂-opioid agonist solus which dosage does not result in the production of analgesia when administered to a human or antinociception when administered to a lower animal requiring alleviation of pain.” The sub-analgesic dosage ranges were defined in terms of commonly accepted lower thresholds for opioids that results in production of analgesia in human adults given by various routes of administration.

Bolan et al. (Journal of Pharmacology and Experimental Therapeutics, 2002, 303(2), 557-562) studied the combination of L-methadone and morphine with various other opioid receptor agonists in mice. It was reported that L-methadone exhibited synergy with morphine, morphine-6-β-glucuronide, codeine, and 6-acetylmorphine. However, both L-methadone and morphine displayed only additive effects with oxymorphone, oxycodone, fentanyl, alfentayl or meperidine.

Grach et al. (British Journal of Pharmacology, 2004) evaluated a combination of morphine and oxycodone in a ratio of 1 to 1 (by weight) dosed at 0.5 mg/kg of body weight versus morphine at 0.5 mg/kg of body weight and oxycodone at 0.5 mg/kg of body weight in a clinical pharmacodynamic setting with healthy subjects in a model of thermal pain. Comparisons of pain magnitude and side effects failed to show any significant differences between the three treatments. The authors concluded that at the doses and ratio tested, the co-administration of morphine and oxycodone did not produce synergistic antinociceptive effects or reduced CNS side effects in healthy humans in the cold pain model.

Clearly there is a need for a preferred range of ratios of morphine to oxycodone containing products for a wide population of patients that provides the best balance between side effect reduction and analgesia.

### SUMMARY OF THE INVENTION

The present invention provides pharmaceutical compositions comprising morphine and oxycodone, or pharmaceutically acceptable salts thereof, in preferred ratios that provide superior analgesia and fewer side effects when compared to doses of the individual components that provide analgesic efficacy.

The pharmaceutical compositions of the invention comprise ratios of morphine to oxycodone of from about 3 to 2 (by weight) to about 1 to 2 (by weight). These ratios of morphine and oxycodone provide the greatest relief of pain at the lowest dose with improved side effect profiles.

In one embodiment, the invention provides an analgesic pharmaceutical composition for oral administration comprising morphine and oxycodone, or pharmaceutically acceptable salts thereof, in a ratio of from about 1.5:1 (3 to 2) to about 0.5:1 (1 to 2), morphine to oxycodone by weight, optionally in combination with a pharmaceutically acceptable carrier. In some embodiments, the pharmaceutical compositions of the invention comprise pharmaceutically acceptable salts of morphine and oxycodone including, but not limited to, salts formed with sulfuric or hydrochloric acid.

In various embodiments, the pharmaceutical compositions of the invention for oral administration comprise morphine and oxycodone in ratios of about 1.5:1, about 0.5:1, about 1.5:1 to about 1.1:1, about 1.5:1 to about 1.2:1, about 0.9:1 to about 0.5:1, or about 0.7:1 to about 0.5:1. In one preferred embodiment, the pharmaceutical compositions for oral administration comprise morphine and oxycodone in a ratio of 1.5:1, morphine to oxycodone by weight. In another preferred embodiment, the pharmaceutical compositions for oral administration comprise a ratio of morphine to oxycodone of 0.5:1, morphine to oxycodone by weight.

In one aspect, the pharmaceutical compositions of the invention are in the form of a solid oral dosage form. The oral dosage forms may be in an immediate release formulation or in the form of a controlled release formulation.

In some embodiments, the pharmaceutical compositions may be in the form of a tablet or capsule. In other embodiments, the pharmaceutical composition may be in a liquid oral dosage form.

In another embodiment, the invention provides pharmaceutical compositions suitable for intravenous or sub-cutaneous administration comprising morphine and oxycodone, or pharmaceutically acceptable salts thereof, in a ratio of about 3 to 2 to about 1.1 to 1, or about 0.9 to 1 to about 0.5 to about 1, morphine to oxycodone by weight.

Also provided are methods for the alleviation of pain in a patient comprising administering to the patient an analgesic pharmaceutical composition for oral administration comprising morphine and oxycodone, or pharmaceutically acceptable salts thereof, in a ratio of from about 1.5:1 (3 to 2) to about 0.5:1 (1 to 2), morphine to oxycodone by weight, optionally in combination with a pharmaceutically acceptable carrier.

In some embodiments, the methods of the invention may comprise combinations of morphine to oxycodone in ratios of about 1.5:1 (3 to 2), about 0.5:1 (1 to 2), about 1.5:1 to about 1.1:1, about 1.5:1 to about 1.2:1, about 0.9:1 to about 0.5:1, or about 0.7:1 to about 0.5:1.

In other embodiments, the invention provides methods for the alleviation of pain in a patient comprising administering to the patient an analgesic pharmaceutical composition for intravenous or sub-cutaneous administration comprising morphine to oxycodone in rations of about 1.5:1 to about 1.1:1, about 1.5:1 to about 1.2:1, about 0.9:1 to about 0.5:1, or about 0.7:1 to about 0.5:1, morphine to oxycodone by weight.

Any suitable route of administration may be employed for providing a human or lower animal the composition of the invention. For example oral, rectal, parenteral, sublingual, buccal, intravenous, intraarticular, intramuscular, intradermal, subcutaneous, inhalational, intraocular, intraperitoneal, epidural, intracerebroventricular, transdermal and the like may be employed.

The present invention may be understood more readily by reference to the following detailed description of the specific embodiments included herein. However, although the present invention has been described with reference to specific details of certain embodiments thereof, it is not intended that such details should be regarded as limitations upon the scope of the invention. The entire text of the references mentioned herein are hereby incorporated in their entirety by reference.

### DETAILED DESCRIPTION

The present invention comprises pharmaceutical compositions and methods comprising morphine and oxycodone for alleviating pain in a patient that provide an optimal analgesic efficacy while minimizing the incidence of undesired opioid side effects. The pharmaceutical compositions of the invention comprise morphine and oxycodone, or pharmaceutically acceptable salts thereof, in a ratio of approximately 1.5:1 (3 to 2) to about 0.5:1 (1 to 2), morphine to oxycodone by weight. These ratios of morphine and oxycodone provide the greatest relief of pain at the lowest dose and with the best side effect profile. The optimal ratios of the dual opioid compositions are determined in part by pharmacokinetic (PK) and pharmacodynamic (PD) profiles from patients or from patient groups treated with morphine and oxycodone combinations.

All ranges disclosed herein are to be understood to encompass any and all subranges subsumed therein. For example, a range of “1.5 to 1” includes any and all subranges between (and including) the minimum value of 1 and the maximum value of 1.5, that is, any and all subranges having a minimum value of equal to or greater than 1 and a maximum value of equal to or less than 1.5, e.g., 1.5 to 1.1.

Where numeral number or range is modified by the term “about,” it will be understood to embrace somewhat larger or smaller values than the indicated value to account for experimental errors inherent in the measurement and variability between different methodologies for measuring the value, as will be apparent to one skilled in the art.

As discussed above, U.S. Pat. No. 6,310,072 to Smith et al. ('072 patent), describes analgesic compositions comprising sub-analgesic doses of morphine and oxycodone. The administration of certain compositions comprising morphine and oxycodone to male Sprague-Dawley rats by intracerebroventricular (i.c.v.), or male Dark Agouti rats by intraperitoneal (i.p.) or subcutaneous (s.c.) administration resulted in synergistic analgesic efficacy. Further, it was reported that rats that received compositions of sub-analgesic doses of morphine and oxycodone approximating the ED₅₀ values of each of the morphine/oxycodone combinations via subcutaneous administration did not exhibit certain adverse side effects compared to rats that received doses approximating the ED₅₀ of morphine or oxycodone alone.

Notwithstanding the efficacy of compositions comprising morphine and oxycodone demonstrated in rats as described in the '072 patent, it is known that the oxycodone and morphine have different intrinsic antinociceptive potencies in rats and humans (see for example Ross et al., Pain 73 (1997), 151-157), and synergistic efficacy in one animal model is not predictive of efficacy in human patients. Additionally, it has been reported that certain combinations of morphine and oxycodone in another animal model did not result in synergistic efficacy and exhibited only additive effects (Bolan et al., The Journal of Pharmacology and Experimental Therapeutics, 2002, 303(2), 557). For example, Bolan et al. reported that morphine in combination with L-methadone exhibited synergistic analgesic efficacy in male CD-1 mice, but that morphine in combination with oxymorphone, oxycodone, fentanyl, alfentanyl or meperidine displayed only additive effects.

In contrast to the synergistic efficacy of combinations of morphine and oxycodone described in the '072 patent in male Sprague-Dawley and male Dark Agouti rats, Grach and co-workers reported that the combination of morphine and oxycodone in a 1:1 weight ratio administered orally to humans has been reported to not yield a synergistic analgesic efficacy in a cold pain model (Grach et al., British Journal of Clinical Pharmacology, 2004). Administration of 0.25 mg/kg morphine sulfate in combination with 0.25 mg/kg oxycodone hydrochloride via oral administration compared 0.5 mg/kg morphine sulfate alone or 0.5 mg/kg oxycodone hydrochloride alone failed to shown synergistic analgesic behavior when administered to human volunteers exposed to an experimental model of cold pressor test (CPT). These reports highlight the complex and unpredictable nature of the cross-reactivity of opioid receptor agonists.

The ratios of morphine to oxycodone of the present invention relate to the weight ratio of the parent compounds in the neutral state. However, it will be apparent to one of skill in the art that the compositions of the invention may comprise pharmaceutically acceptable salts of the compounds as long as the salts are present in an amount that corresponds to the desired weight ratio of the parent compounds. The pharmaceutically acceptable salts of morphine and oxycodone may be prepared with any pharmaceutically acceptable acid including, but not limited to, sulfuric acid and hydrochloric acid.

The pharmaceutical compositions may be in the form of solid or liquid dosage forms including tablets, capsules, dispersions, suspensions, injections, solutions, syrups, troches, capsules, suppositories, aerosols, transdermal patches and the like. These dosage forms may also include compositions implanted in a patient that are designed to provide sustained release of the active agents. Controlled release of the opioids may be affected by incorporating the opioids into, for example, hydrophobic polymers including acrylic resins, waxes, higher aliphatic alcohols, polylactic and polyglycolic acids and certain cellulose derivatives such as hydroxypropylmethyl cellulose. In addition, the controlled release may be affected by using other polymer matrices, liposomes and/or microspheres.

Pharmaceutically-acceptable carriers for systemic administration may also be incorporated into the compositions of this invention. By “pharmaceutically-acceptable carrier” is meant a solid or liquid filler, diluent or encapsulating substance which may be safely used in systemic administration. Depending upon the particular route of administration, a variety of pharmaceutically-acceptable carriers, well known in the art may be used. These carriers include sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

In one embodiment, the invention provides pharmaceutical compositions comprising morphine and oxycodone, or pharmaceutically acceptable salts thereof, in a ratio of about 1.5:1 to about 0.5:1, morphine to oxycodone by weight. In other embodiments, the invention provides pharmaceutical compositions comprising morphine and oxycodone, or pharmaceutically acceptable salts thereof, in a ratio of about 1.5:1 to about 0.75:1, about 1.5:1 to about 1:1, or about 1.5:1 to about 1.1:1. In other embodiments, morphine and oxycodone are provided in ratios of about 1.5:1 to about 1.2:1, about 1.5:1 to about 1.3:1, or about 1.5:1 to about 1.4:1, morphine to oxycodone by weight.

In still other embodiments, the invention provides pharmaceutical compositions comprising morphine and oxycodone, or pharmaceutically acceptable salts thereof, in a ratio of about 1.25:1 to about 0.5:1, about 1:1 to about 0.5:1, about 0.9:1 to about 0.5:1, about 0.8:1 to about 0.5:1, about 0.7:1 to about 0.5:1, or about 0.6:1 to about 0.5:1.

In one preferred embodiment, the invention provides pharmaceutical compositions comprising morphine and oxycodone in a ratio of about 1.5:1, morphine to oxycodone by weight. In another preferred embodiment, the invention provides pharmaceutical compositions comprising morphine and oxycodone in a ratio of about 0.5:1, morphine to oxycodone by weight.

In one embodiment, the compositions of the invention may be administered by intravenous or sub-cutaneous administration. The sterile injectable preparation may be a sterile injectable solution or suspension in a non-toxic parenterally-acceptable diluent or carrier known in the art for intravenous or sub-cutaneous administration, for example as a solution in water, physiological saline, Ringer's solution, or phosphate buffered saline (PBS), or in suitable non-aqueous carriers.

In a particular embodiment, the invention provides a composition comprising morphine and oxycodone, or pharmaceutically acceptable salts thereof, suitable for intravenous or sub-cutaneous administration, wherein the morphine and oxycodone are in a ratio of about 1.5:1 to about 1.1:1, about 1.5:1 to about 1.2:1, about 1.5:1 to about 1.3:1, or about 1.5:1 to about 1.4:1, morphine to oxycodone by weight.

In other embodiments, the invention provides pharmaceutical compositions comprising morphine and oxycodone, or pharmaceutically acceptable salts thereof, suitable for intravenous or sub-cutaneous administration in a ratio of about 0.9:1 to about 0.5:1, about 0.8:1 to about 0.5:1, about 0.7:1 to about 0.5:1, or about 0.6:1 to about 0.5:1.

In one preferred embodiment, the invention provides pharmaceutical compositions comprising morphine and oxycodone suitable for intravenous or sub-cutaneous administration in a ratio of about 1.5:1, morphine to oxycodone by weight. In another preferred embodiment, the invention provides pharmaceutical compositions comprising morphine and oxycodone suitable for intravenous or sub-cutaneous administration in a ratio of about 0.5:1, morphine to oxycodone by weight.

In another aspect of the invention, pharmaceutical compositions comprising morphine and oxycodone in formulations that are suitable for oral administration are provided. Pharmaceutical compositions suitable for oral administration may be in the form of solid oral dosage forms or liquid oral dosage forms and may include pharmaceutically acceptable carriers and excipients known in the art. Suitable vehicles and their formulation are described, for example, in Remington: The Science and Practice of Pharmacy, 21st ed, Lippincott Williams & Wilkins (2005).

The concentration of morphine and oxycodone in the blood stream will depend on the amount of compound administered in the composition as well as the method of administration and the specific formulation used. For example, it is well known in the art that administration of morphine and oxycodone by intravenous injection typically results in a significant concentration of each compound in the blood stream substantially immediately after administration (without delay), whereas formulations adapted for oral administration of morphine and oxycodone will typically achieve effective concentrations in the blood stream later than intravenous administration and at different concentrations depending on oral availability of the compounds. Further, the means of administration of the compounds may result in different inactivation and excretion rates of morphine and oxycodone when administered in a combination. Therefore, it will be apparent to one of skill in the art that the absolute and relative amounts of morphine and oxycodone administered to patients via oral administration to achieve a synergistic efficacy with a lower incidence of adverse side effects will differ from the amounts of drugs required for intravenous administration.

In one embodiment, the invention provides pharmaceutical compositions suitable for oral administration comprising morphine and oxycodone, or pharmaceutically acceptable salts thereof, in a ratio of about 1.5:1 to about 0.5:1, morphine to oxycodone by weight, that provide the greatest relief of pain at the lowest dose and an improved side effect profile. The oral pharmaceutical compositions provide analgesic efficacy at a lower total drug dose by weight compared to the doses of either morphine or oxycodone alone required to achieve analgesic efficacy. Additionally, the oral pharmaceutical compositions of the invention alleviate pain with a lower incidence of undesired opioid-related side effects compared with morphine or oxycodone alone.

The pharmaceutical compositions for oral administration may be in the form of immediate release or controlled release formulations. Pharmaceutical compositions comprising immediate release or controlled release formulations of opioid drugs are well known in the art. For example, OXYIR® is an immediate release formulation of oxycodone and OXYCONTIN® is a controlled release formulation of oxycodone, which provides controlled delivery of oxycodone over 12 hours. Similarly, MS CONTIN® is a controlled release formulation of morphine sulfate and MSIR® is an immediate release formulation of morphine sulfate.

Immediate release formulations of pharmaceutical compositions will typically comprise ingredients that break down quickly after administration to release the active components. In some embodiments, the pharmaceutical compositions of the invention in immediate release formulations will exhibit a Tₘₐₓ, the time required for a compound to reach its maximum concentration in circulation (Cₘₐₓ), of from about 10 minutes to about 2 hours after ingestion. In other embodiments, the Tₘₐₓ will be from about 10 minutes to about 1 hour, about 10 minutes to about 30 minutes or about 10 minutes to about 45 minutes.

The pharmaceutical compositions in controlled release or sustained release formulations will typically exhibit longer Tₘₐₓ times and display significant concentrations in circulation for longer periods of time. In some embodiments, the Tₘₐₓ of controlled release formulations of the invention will be from about 1 hour to about 4 hours, from about 1 hour to about 3 hours, from about 1 hour to about 2.5 hours or from about 1 hour to about 2 hours.

The controlled release formulation of morphine and oxycodone will be released at a slower rate and over a longer period of time. For example, in some embodiments, the controlled release formulation of morphine and oxycodone will release effective amounts of a mixture of morphine and oxycodone over 12 hours. In other embodiments, the controlled release formulation will release effective amounts of morphine and oxycodone over 4 hours or over 8 hours. In still other embodiments, the controlled release formulation will release effective amounts of morphine and oxycodone over 15, 18, 24 or 30 hours.

In other embodiments, the invention provides pharmaceutical compositions suitable for oral administration comprising morphine and oxycodone, or pharmaceutically acceptable salts thereof, in a ratio of about 1.5:1 to about 0.75:1, about 1.5:1 to about 1:1, or about 1.5:1 to about 1.1:1. In other embodiments, morphine and oxycodone are provided in ratios of about 1.5:1 to about 1.2:1, about 1.5:1 to about 1.3:1, or about 1.5:1 to about 1.4:1, morphine to oxycodone by weight.

In still other embodiments, the invention provides pharmaceutical compositions suitable for oral administration comprising morphine and oxycodone, or pharmaceutically acceptable salts thereof, in a ratio of about 1.25:1 to about 0.5:1, about 1:1 to about 0.5:1, about 0.9:1 to about 0.5:1, about 0.8:1 to about 0.5:1, about 0.7:1 to about 0.5:1, or about 0.6:1 to about 0.5:1

In one preferred embodiment, the invention provides pharmaceutical compositions suitable for oral administration comprising morphine and oxycodone in a ratio of about 1.5:1, morphine to oxycodone by weight. In another preferred embodiment, the invention provides pharmaceutical compositions suitable for oral administration comprising morphine and oxycodone in a ratio of about 0.5:1, morphine to oxycodone by weight.

Another aspect of this invention relates to a method of alleviating pain in a patient, which method comprises administering to a patient in need of such a treatment a composition comprising morphine and oxycodone, or pharmaceutically acceptable salts thereof, in a ratio of approximately 1.5:1 to approximately 0.5:1 (morphine to oxycodone by weight). The methods and compositions of the invention may be used to treat acute or chronic pain, including neuropathic pain or nociceptive pain. Further, mixed pain states comprising neuropathic pain and nociceptive pain may be effectively treated.

In other embodiments, the invention provides methods of alleviating pain in a patient, which methods comprise administering to the patient a combination of morphine and oxycodone, or pharmaceutically acceptable salts thereof, in a ratio of about 1.5:1 to about 0.75:1, about 1.5:1 to about 1:1, or about 1.5:1 to about 1.1:1. In other embodiments, morphine and oxycodone are provided in ratios of about 1.5:1 to about 1.2:1, about 1.5:1 to about 1.3:1, or about 1.5:1 to about 1.4:1, morphine to oxycodone by weight.

In other embodiments, the invention provides methods of alleviating pain in a patient comprising administering to the patient a combination of morphine and oxycodone, or pharmaceutically acceptable salts thereof, in a ratio of about 1.25:1 to about 0.5:1, about 1:1 to about 0.5:1, about 0.9:1 to about 0.5:1, about 0.8:1 to about 0.5:1, about 0.7:1 to about 0.5:1, or about 0.6:1 to about 0.5:1.

The invention also provides uses of morphine and oxycodone, or pharmaceutically acceptable salts thereof, in a ratio of about 1.5 to 1 to about 1 to 2, morphine to oxycodone by weight, in the treatment of pain in a patient or in the manufacture of a medicament for the treatment of pain in a patient.

In other embodiments, the invention provides uses of morphine and oxycodone, or pharmaceutically acceptable salts thereof, in a ratio of about 1.5:1 to about 0.75:1, about 1.5:1 to about 1:1, or about 1.5:1 to about 1.1:1. In other embodiments, morphine and oxycodone are provided in ratios of about 1.5:1 to about 1.2:1, about 1.5:1 to about 1.3:1, or about 1.5:1 to about 1.4:1, morphine to oxycodone by weight, in the treatment of pain in a patient or in the manufacture of a medicament for the treatment of pain in a patient.

In other embodiments, the invention provides uses of morphine and oxycodone, or pharmaceutically acceptable salts thereof, in a ratio of about 1.25:1 to about 0.5:1, about 1:1 to about 0.5:1, about 0.9:1 to about 0.5:1, about 0.8:1 to about 0.5:1, about 0.7:1 to about 0.5:1, or about 0.6:1 to about 0.5:1, morphine to oxycodone by weight, in the treatment of pain in a patient or in the manufacture of a medicament for the treatment of pain in a patient.

In preferred embodiments, the invention provides methods of alleviating pain in a patient, which methods comprise administering to the patient a pharmaceutical composition in the form of an oral dosage form comprising a combination of morphine and oxycodone, or pharmaceutically acceptable salts thereof, in a ratio of about 1.5:1 to about 0.5:1, morphine to oxycodone by weight. In other embodiments, the invention provides methods of alleviating pain in a patient comprising administering to the patient a pharmaceutical composition in the form of an oral dosage form comprising a combination of morphine and oxycodone, or pharmaceutically acceptable salts thereof, in a ratio of about 1.5:1 to about 0.75:1, about 1.5:1 to about 1:1, or about 1.5:1 to about 1.1:1. In other embodiments, morphine and oxycodone are provided in ratios of about 1.5:1 to about 1.2:1, about 1.5:1 to about 1.3:1, or about 1.5:1 to about 1.4:1, morphine to oxycodone by weight.

In still other embodiments, the invention provides methods of alleviating pain in a patient comprising administering to the patient a pharmaceutical composition in the form of an oral dosage form comprising a combination of morphine and oxycodone, or pharmaceutically acceptable salts thereof, in a ratio of about 1.25:1 to about 0.5:1, about 1:1 to about 0.5:1, about 0.9:1 to about 0.5:1, about 0.8:1 to about 0.5:1, about 0.7:1 to about 0.5:1, or about 0.6:1 to about 0.5:1.

The absolute amounts of morphine and oxycodone present in the pharmaceutical compositions of the invention or administered in the methods of the invention are not limited and will be determined for a particular patient based on the specific needs of the patient and the type of pain being treated. The specific dose will depend upon a variety of patient-specific factors, including, but not limited to, the age, the body weight, general health, sex, diet, time of administration, rate of excretion, and the judgment of the treating physician and the severity of the condition that requires analgesic treatment. Further, the amount of the active compounds that may be combined with the carrier materials to produce a single dosage form will vary depending upon the patient treated, the purpose of treatment, the pain state and whether the patient is opioid-naïve or has developed tolerance. The particular mode of administration will also affect the dose of the compound given to a patient.

In some embodiments of the invention, an initial dose of the morphine component, or pharmaceutically acceptable salts thereof, in the pharmaceutical compositions comprising morphine and oxycodone or methods of the invention for a näive human adult through a subcutaneous, intravenous, intramuscular, buccal or sublingual route may be between about 0.5 mg and about 20 mg, about 0.5 mg and about 15 mg, or about 0.5 mg and about 10 mg. In other embodiments, the dose will be between about 0.5 mg and about 5 mg, about 0.5 mg and about 3.5 mg, between about 0.5 mg and about 3.0 mg, between about 0.5 mg and about 2.5 mg, or between about 0.5 mg and about 2.0 mg every four hours.

In other embodiments, an initial dose of the morphine component in a controlled-release dosage form comprising morphine and oxycodone may be between about 1 mg to about 200 mg, about 1 mg to about 100 mg, about 1 mg to about 50 mg or about 1 mg to about 20 mg. In other embodiments, the initial dose of the morphine component in a controlled-release dosage form may be between about 1 mg and about 15 mg, about 1 mg and about 10 mg, between about 1 mg and about 9 mg, between about 1 mg and about 7.5 mg or between about 1 mg and about 6 mg every 12 hours, or between about 3 mg and about 21 mg, between about 3 mg and about 18 mg, between about 3 mg and about 15 mg or between about 3 mg and about 12 mg every 24 hours. In other embodiments comprising controlled-release dosage forms, the initial dose of the morphine component may be between about 6 mg and about 75 mg, between about 15 mg and about 60 mg, between about 15 mg and about 45 mg every 12 hours, or between about 12 mg and about 150 mg, between about 30 mg and about 120 mg, or between about 30 mg and about 90 mg every 24 hours.

In other embodiments, an initial dose of the morphine component, or pharmaceutically acceptable salts thereof, in the pharmaceutical compositions of the invention for a näive human adult through an oral or rectal route is between about 1 mg to about 200 mg, about 1 mg to about 100 mg, or about 1 mg to about 50 mg. In other embodiments, the initial dose of the morphine component in a controlled-release dosage form may be between about 2 mg and about 25 mg, between about 5 mg and about 20 mg, or between about 5 and about 15 mg every four hours.

In still other embodiments of the invention, an initial dose of the morphine in the pharmaceutical compositions comprising morphine and oxycodone and methods of the invention for a human adult through an intracerebroventricular route may be between about 0.05 mg and about 0.25 mg per day.

In other embodiments, an initial dose of the oxycodone component, or derivative or pharmaceutically acceptable salts thereof, in the pharmaceutical compositions comprising morphine and oxycodone and methods of the invention for a naive human adult through a subcutaneous or intravenous route may be between 0.5 mg and about 20 mg, about 0.5 mg and about 15 mg, or about 0.5 mg and about 10 mg. In other embodiments, the initial dose of the oxycodone component of the compositions may be about 1 mg and about 8 mg, between about 1 mg and about 6 mg, or between about 1 and about 4 mg every four hours.

In some embodiments comprising controlled-release dosage forms, an initial dose of the oxycodone component in the compositions of the invention may be between about 1 mg to about 200 mg, about 1 mg to about 100 mg, about 1 mg to about 50 mg or about 1 mg to about 20 mg. In other embodiments, the initial dose of the oxycodone component may be between about 3 mg and about 24 mg, between about 3 mg and about 18 mg, between about 3 mg and about 12 mg every 12 hours, or between about 6 mg and about 48 mg, between about 6 mg and about 36 mg, or between about 6 mg and about 24 mg every 24 hours.

In additional embodiments, an initial dose of the oxycodone component, or pharmaceutically acceptable salts thereof, in the pharmaceutical compositions comprising morphine and oxycodone and methods of the invention for a näive human adult through an oral or rectal route is between about 1 mg to about 200 mg, about 1 mg to about 100 mg, or about 1 mg to about 50 mg. In other embodiments, the initial dose of the oxycodone component may be between about 1 mg to about 25 mg, about 1 mg to about 15 mg, about 1 mg to about 10 mg, about 1 mg and about 8 mg, between about 1 mg and about 6 mg, or between about 1 and about 4 mg every four hours. Alternatively, in some embodiments comprising controlled-release dosage forms, the initial dose of the oxycodone component may be between about 3 mg and about 24 mg, between about 3 mg and about 18 mg, between about 3 mg and about 12 mg every 12 hours, or between about 6 mg and about 48 mg, between about 6 mg and about 36 mg, or between about 6 mg and about 24 mg every 24 hours.

Following extensive studies investigating the varying impacts of administering therapeutic opioid compositions to human patients in order to alleviate pain symptoms while minimizing opioid-related adverse side effects, the present inventors determined that the preferred range of ratios of morphine and oxycodone in dual opioid compositions is about 1.5:1 (3 to 2) to about 0.5:1 (1 to 2), morphine to oxycodone by weight.

The studies are detailed in the Examples below, however in summary the inventors primarily tested Q8001 (morphine and oxycodone in approximately 3 to 2 ratio, morphine to oxycodone by weight), Q8002 (morphine/oxycodone in approximately 1:2 ratio, morphine to oxycodone by weight) and Q8004 (morphine alone) in comparative studies to evaluate relative pain relief and safety on patients with chronic moderate to severe, non-cancerous pain. Combinations of morphine and oxycodone in the optimal ranges were also evaluated for the treatment of acute pain following bunionectomy surgery. In addition to evaluating pain alleviation, the incidence and severity of adverse side effects such as nausea, constipation, drowsiness and vomiting was evaluated.

The present invention will be understood more readily by reference to the following examples, which are provided by way of illustration and are not intended to be limiting of the invention.

### EXAMPLE 1

Comparison of a Product Containing Morphine and Oxycodone in the Ratio of 3 to 2, Morphine to Oxycodone by Weight vs. Morphine Alone (Study 1) and a Product Containing Morphine and Oxycodone in the Ratio of 1 to 2, Morphine to Oxycodone by Weight vs. Morphine Alone (Study 2) in the Treatment of Chronic Non-Cancer Pain

### Study Design

Both Study 1 and Study 2 were Phase II clinical trials in patients with chronic moderate to severe, non-cancer pain. Both designs were similar, in that they were randomized, double-blind, crossover studies in an inpatient facility for up to a seven day period, for each arm. A standard comparison of each treatment was morphine in both studies. The primary objectives of these protocols were to compare pain relief and safety attributable to either of the two treatment groups. Efficacy was assessed using a Visual Analogue Scale (VAS) pain based on a patient self assessment.

### Efficacy

Study 1 was conducted in 21 patients with chronic non-cancer pain who were receiving opioid drugs for pain management prior to entry into the study. The study tested a 3 to 2 by weight combination of morphine and oxycodone in an oral liquid formulation designated Q8001, against morphine (Q8004) alone. In the 21 evaluable patients a steady state pain control dose was achieved with both dose formulations, but 49% drug by weight were needed when Q8001 was administered.

A second crossover study (Study 2) was similar in design to Study 1. Twenty-three patients with chronic non-cancer pain were studied using a 1 to 2 morphine to oxycodone ratio designated Q8002. Also a dose reduction of 49% by weight of Q8002 compared to morphine was seen. These data indicated that both Q8001 and Q8002 combination ratios were analgesic. The baseline pain values represented the amount of pain experienced by the patient while receiving opioid therapy. Therefore, the incremental improvements in pain control, while positive, were of a modest magnitude. The essential point of the study was the demonstration of the reduction in the amount of opioid medication needed to achieve a meaningful level of pain management.

TABLE 1
Summary of Efficacy Results from Studies 1 and 2
(mean reduction in Pain (VAS) Scores from Baseline)

| | | PAIN SCORE (VAS) | | |
|---|---|---|---|---|
| | | | | % PAIN |
| Study # | | At BASELINE | At DAYS 4-5 | REDUCTION |
| 1 | MORPHINE | 4.5 | 4 | 11 |
| 1 | Q8001 | 4.6 | 4.2 | 9 |
| 2 | MORPHINE | 4.8 | 3.8 | 21 |
| 2 | Q8002 | 5.4 | 3.8 | 30 |

### Opiate Related Adverse Events

Based on knowledge from clinical pharmacology and prior clinical studies with opioids, four frequently reported opioid-specific adverse events: nausea, vomiting, constipation and drowsiness were selected for repeated inquiry and grading throughout both studies. Assessments were made at baseline (pre-treatment) on Day 1 at 6, 12 and 24 hours, Days 2, 3, 4, 5, 6 and 7.

The severity of nausea was graded on a scale of 0-3, and vomiting, constipation and drowsiness were scored on a graded scale of 0-4.

### Incidence Rates for Selected Adverse Events

A summary of the selected adverse events, for Study 1, is presented in Table 2 below. The scores were transformed into binary events (AE=‘No’ if 0, or no event, AE=‘Yes’ if a graded score of 1 or higher is recorded). Each row in the table represents the relationship between treatments and the risk of experiencing the adverse event listed. The Chi-Square test compares the incident rates between treatment groups, for each selected adverse event (p-values). Further, table 2 shows the Relative Risk of how much more likely patients would report the referenced adverse events if morphine alone is administered rather than the Q8001 combination of morphine and oxycodone.

TABLE 2
Study 1-Incidence Rates (%), Chi-Square and Relative Risk
Determinations (95% Confidence Intervals [CI]) for
Opioid-Related Adverse Events by Treatment Group

| | Morphine | | Q8001 | | Chi-Square | Relative Risk |
|---|---|---|---|---|---|---|
| Side Effects | Yes | No | Yes | No | p-Value | (95% CI) |
| Nausea | 12 | 9 | 6 | 15 | 0.061 | 2.000 |
| | (57%) | (43%) | (29%) | (71%) | | 0.925-4.324 |
| Constipation | 12 | 9 | 10 | 11 | 0.536 | 1.200 |
| | (57%) | (43%) | (48%) | (52%) | | 0.671-2.147 |
| Drowsiness | 15 | 6 | 10 | 11 | 0.111 | 1.500 |
| | (71%) | (29%) | (48%) | (52%) | | (0.888-2.533) |
| Vomiting | 2 | 19 | 2 | 19 | * | * |
| | (10%) | (90%) | (10%) | (90%) | Similar rates | Similar rates |

* Clearly no difference in rates

Study 1 showed that both nausea (p=0.06) and drowsiness (p=0.12) marginally failed to reach the significant level of p<0.05, favoring Q8001 over morphine. These probabilities of expected differences in treatments may be better reflected in the Relative Risk (RR) determinations. For nausea the RR is 2.0 (95% CI 0.93-4.32) and for drowsiness the RR is 1.5 (CI 0.89-2.53). There were no discernible differences between treatments for constipation and vomiting.

A summary of the selected opioid-specific adverse events for Study 2 is presented in Table 3 below. The Chi-Square test compares the incident rates between treatment groups, for each adverse event (p-values). In this study, nausea (p=0.641), drowsiness (p=0.63) and vomiting (p=1.00) do not show a trend favoring Q8002 over morphine. However, the incidence of constipation shows a statistical significant difference (p=0.025) favoring Q8002. This is reflected in the Relative Risk (RR) determination for constipation, where patients in the morphine group may have had up to 4 times the risk (95% upper limit of the RR).

TABLE 3
Study 2-Incidence Rates (%), Chi-Square and Relative Risk
Determinations (95% Confidence Intervals [CI]) for Opioid-Related
Adverse Events by Treatment Group

| | Morphine | | Q8002 | | Chi-Square | Relative Risk |
|---|---|---|---|---|---|---|
| Side Effects | Yes | No | Yes | No | p-Value | (95% CI) |
| Nausea | 129 | 11 | 13 | 9 | 0.641 | 0.883 |
| | (52%) | (48%) | (59%) | (41%) | | (0.523-1.4900 |
| Constipation | 15 | 8 | 7 | 15 | 0.025* | 2.050 |
| | (65%) | (35%) | (32%) | (68%) | | (1.038-4.048) |
| Drowsiness | 10 | 13 | 8 | 14 | 0.626 | 1.197 |
| | (43%) | (57%) | (36%) | (64%) | | (0.580-2.464) |
| Vomiting | 3 | 20 | 3 | 19 | ** | ** |
| | (13%) | (87%) | (14%) | (86%) | Similar rates | Similar rates |

*Statistically different at p < 0.05
** Clearly no difference in rates

### Conclusions

Both Q8001 (morphine to oxycodone ratio of 3 to 2) and Q8002 (morphine to oxycodone ratio of 1 to 2) both gave improved pain relief based on the total dose of drug compared to morphine alone.

Both Q8001 and Q8002 treatment regimens exhibited reductions of certain opioid related side effects compared to morphine alone.

### EXAMPLE 2

### Efficacy and Safety Study of Products Containing Morphine and Oxycodone in a Ratio of 3 to 2 (by Weight) in Acute Pain

### Study Design

This double-blind, ascending cohort, parallel treatment study (Study 3) evaluated analgesia and safety measures in 5 groups of patients with moderate to severe pain (numerical pain rating scale score, range 0-10, study inclusion score of at least 4) following bunionectomy surgery, a procedure that involves manipulation of metatarsal foot bone. Once each of the 256 patients had sufficient pain following surgery, they were randomized to oxycodone/morphine or placebo and received dosing for up to the next 48 hrs. The dosing schedule was flexible in that the inventor wanted to determine what dosing intervals were preferred (and the amount of drug received) by patients as a function of unit dose strengths of products oxycodone and morphine in the ratio of 3 to 2. Dosages administered were 3/2 mg, 6/4 mg, 12/8 mg and 18/12 mg or 0/0 mg (placebo). Patients were also allowed rescue analgesia (600 mg ibuprofen), but following ibuprofen dosing pain data was censored for a period of time. In addition to the pain ratings, a patient rating of overall clinical satisfaction with study medication at the end of dosing was utilized. A total of about 50-60 patients entered into each of the 5 treatment arms. Also collected were plasma samples for post-study measurement of blood levels of morphine and of oxycodone (3 samples per patient over the 48 hr study period).

### PK/PD Approach

Using the data on blood plasma levels of morphine and oxycodone measured at three time points (sparse sampling approach) during the 48 hr dosing period, a pharmacokinetic model was constructed in which the full curve of plasma morphine and of oxycodone levels over a 48 hr period for each patient was generated. The model itself used PK data from prior single dose and repeat dose phase 1 studies of morphine/oxycodone products. Also a definition of patients that received superior pain relief (termed ‘superior responders’) and of patients that received adequate pain relief (termed ‘adequate responders’) was provided using the efficacy data obtained from Study 3. For the PK/PD analysis, a patient with superior pain relief was defined as a patient who at termination of treatment had at least a 30% reduction in pain intensity from baseline and/or a global rating of much to excellent improvement in pain reduction.

PK/PD Data for Patients with Superior Responders Versus Adequate Responders (Data Pooled Across All Dose Levels)

Table 4 below shows key results of PK parameters for superior responders (n=129) versus adequate responders (n=64) patients who received the morphine/oxycodone containing products (placebo data are omitted). The Table summarizes the results for adequate responders and superior responder patients for several PK parameters.

TABLE 4
Summary of PK/PD data for superior responders versus
adequate responders (data pooled across all dose levels).

| | Mean | | | | |
|---|---|---|---|---|---|
| | Adequate | Mean Superior | | | |
| | Responders | Responders | | | Mean: All |
| | (n = 64 pts) | (n = 129) | t-test | p-value | Patients |
| Median Ratio in Plasma | 0.4421 | 0.2793 | 4.80 | <0.001 | 0.3333 |
| Level of Morphine to | | | | | |
| Oxycodone | | | | | |
| Ratio of AUC Morphine | 0.2478 | 0.2347 | 1.57 | 0.119 | 0.2391 |
| to AUC Oxycodone | | | | | |
| Morphine Cmax (nM) | 10.09 | 10.88 | 0.97 | NS | 10.63 |
| Oxycodone Cmax (nM) | 44.18 | 49.08 | 1.44 | 0.152 | 47.45 |
| Morphine AUC | 217.47 | 259.47 | 1.61 | 0.108 | 245.54 |
| (nM * hr) | | | | | |
| Oxycodone AUC | 908.88 | 1144.50 | 2.18 | 0.030 | 1066.37 |
| (nM * hr) | | | | | |

*the median ratio was determined for each patient across all time points. The t-tests compared the mean of the median ratios for superior responders vs. adequate responders.

### Results and Conclusions

As shown above, the only parameter that showed a pronounced statistically significant difference between superior responders and adequate responders was the ratio of the mean plasma level of morphine to oxycodone. The lower ratios of the mean plasma levels of morphine to oxycodone in the responder group means that patients with a higher proportion of oxycodone to morphine in the blood tended to have a better response. The superior responders had an oxycodone to morphine ratio that was about 1.6 times that of the adequate responders. The ratio of the areas under the curve (AUCs), which takes into account the magnitude of the plasma concentrations at each time point, showed a non-significant trend in the same direction, for example, higher proportions of oxycodone to morphine gave a better effect. This non-significant trend is consistent with the observation that absolute amounts of drug in blood were not strongly related to outcome.

Based on the investigations detailed herein, it is projected that formulations containing morphine plus oxycodone in a ratio of 3 to 2 or less (by weight) will produce a plasma ratio of morphine to oxycodone levels of approximately 0.33 or less during repeat dosing produces and thus will yield a significantly better therapeutic effect compared to formulations containing morphine plus oxycodone in a ratio of less than or equal to 3 to 2 by weight. This prediction is in-line with the results of Studies 1 and 2 presented above in which based on pain reduction compared with its morphine control the product used in Study 2 containing morphine to oxycodone in a ratio of 1 to 2 gave better efficacy than the product used in Study 1 containing morphine to oxycodone in a ratio of 3 to 2.

### EXAMPLE 3

Comparison of a Product Containing Morphine and Oxycodone in the Ratio of 3 to 2 vs. Morphine vs. Oxycodone (Study 4) in the Treatment of Acute Pain

The purpose of Study 4 was to compare the efficacy and opioid related adverse events of products containing morphine and oxycodone in the ratio of 3 to 2 by weight with its individual components.

### Design and Main Measures

Study 4 was a double-blind, randomized, multicenter, 48 hr treatment duration, bunionectomy study. The fixed dose treatment arms (q6h) were (i) products containing morphine and oxycodone as follows: 12 mg/8 mg and 6 mg/4 mg, and (ii) 4 products containing the components alone as follows: morphine 12 mg, morphine 6 mg, oxycodone 8 mg and oxycodone 4 mg. Pain reduction over a 24 hr period (SPID24: area under the curve of the changes in pain from baseline for the 24 hr period following the first dose of study medication) was prospectively defined as the primary endpoint. Also, in this trial ibuprofen rescue medication was 400 mg and was limited to a total dose of 3200 mg per 24 hrs. A total of 197 patients were enrolled in the study. A pain relief equivalency ratio of 1.5 parts of morphine is equivalent to one part of oxycodone was used in determining ‘morphine equivalents’ for comparison purposes (see Table 5).

TABLE 5
Morphine Equivalent Doses of Product Used in Study 4

| Product Containing Morphine to | | |
|---|---|---|
| Oxycodone in the Ratio of 3 to 2 | Morphine- | Oxycodone- |
| (by weight) | Equivalent Dose | Equivalent Dose |
| Product: 6 mg/4 mg | 12 mg | 8 mg |
| Product: 12 mg/8 mg | 24 mg* | 16 mg* |

*These doses were not studied in the current study

### Results

The key safety results from the study are shown in Table 6 (opioid related adverse events).

When comparing equi-analgesic doses (i.e. doses with of the same morphine equivalents) the opioid adverse event (AE) incidence for the 6 mg/4 mg product (which is the morphine equivalent dose of 12 mg morphine and of 8 mg oxycodone) had much lower rates of moderate to severe nausea, vomiting, dizziness and constipation compared to the monotherapy groups (Table 6). There were 20-75% decreases in most of these event rates in the 6 mg/4 mg product. Also the overall tolerance as measured by the number of patients dropping out during the study was better in the combination product compared to its individual components.

TABLE 6
Comparative AE Percents for the Most Common Opioid Events
at Morphine Equivalent Doses

| | Morphine & | Morphine | Oxycodone |
|---|---|---|---|
| Product | Oxycodone 6/4 mg | 12 mg | 8 mg |
| Morphine Equivalent | 12 mg | 12 mg | 12 mg |
| of Product | | | |
| Number of Patients | 32 | 29 | 34 |
| per Group | | | |
| Adverse Events (%) | | | |
| Nausea | 44 | 59 | 50 |
| mild/mod/severe | | | |
| Nausea - | 9 | 28 | 26 |
| moderate/severe | | | |
| Vomiting | 6 | 21 | 21 |
| mild/mod/severe | | | |
| Vomiting - | 6 | 21 | 21 |
| moderate/severe | | | |
| Dizziness | 13 | 31 | 24 |
| mild/mod/severe | | | |
| Dizziness - | 3 | 7 | 12 |
| moderate/severe | | | |
| Constipation | 3 | 10 | 6 |
| mild/mod/severe | | | |
| Constipation - | 3 | 10 | 6 |
| moderate/severe | | | |
| Dropouts (%) | 3 | 7 | 12 |

It is to be understood that this invention is not limited to the particular combinations, methods, and materials disclosed herein as such combinations, methods, and materials may vary somewhat. It is also to be understood that the terminology employed herein is used for the purpose of describing particular embodiments only and is not intended to be limiting.

1. An analgesic pharmaceutical composition for oral administration comprising morphine and oxycodone, or pharmaceutically acceptable salts thereof, in a ratio of about 3 to 2 morphine to oxycodone by weight, optionally in combination with a pharmaceutically acceptable carrier, wherein the morphine and the oxycodone are present in amounts of about 3-200 mg morphine and about 2-200 mg oxycodone.

2. The pharmaceutical composition of claim 1, wherein the composition comprises pharmaceutically acceptable salts of morphine and oxycodone.

3-8. (canceled)

9. The pharmaceutical composition of claim 1, wherein the composition is in the form of a solid oral dosage form.

10. The pharmaceutical composition of claim 9, wherein the dosage form comprises an immediate release formulation.

11. The pharmaceutical composition of claim 9, wherein the dosage form comprises a controlled release formulation.

12. The pharmaceutical composition of claim 9, wherein the dosage form is a tablet or capsule.

13. The pharmaceutical composition of claim 1, wherein the dosage form is a liquid oral dosage form.

14. An analgesic pharmaceutical composition for intravenous or sub-cutaneous administration comprising morphine and oxycodone, or pharmaceutically acceptable salts thereof, in a ratio of about 3 to 2 morphine to oxycodone by weight, optionally in combination with a pharmaceutically acceptable carrier, wherein the morphine and the oxycodone are present in amounts of about 3-200 mg morphine and about 2-200 mg oxycodone.

15. (canceled)

16. A method for the alleviation of pain in a patient comprising administering to the patient the pharmaceutical composition of claim 1.

17-22. (canceled)

23. A method for the alleviation of pain in a patient comprising administering to the patient the pharmaceutical composition of claim 14.

24-32. (canceled)

33. The analgesic pharmaceutical composition of claim 1, wherein the composition comprises about 3 mg morphine and about 2 mg oxycodone.

34. The analgesic pharmaceutical composition of claim 1, wherein the composition comprises about 6 mg morphine and about 4 mg oxycodone

35. The analgesic pharmaceutical composition of claim 1, wherein the composition comprises about 12 mg morphine and about 8 mg oxycodone.

36. The analgesic pharmaceutical composition of claim 1, wherein the composition comprises about 18 mg morphine and about 12 mg oxycodone.

37. An analgesic pharmaceutical composition for oral administration comprising morphine and oxycodone, or pharmaceutically acceptable salts thereof, optionally in combination with a pharmaceutically acceptable carrier, wherein the morphine and the oxycodone are present in amounts of about 6 mg morphine and about 4 mg oxycodone.

38. An analgesic pharmaceutical composition for oral administration comprising morphine and oxycodone, or pharmaceutically acceptable salts thereof, optionally in combination with a pharmaceutically acceptable carrier, wherein the morphine and the oxycodone are present in amounts of about 12 mg morphine and about 8 mg oxycodone.
